(19) United States
(12) Patent Application Publication
(10) Pub. No.: US 2024/0261086 A1
(43) Pub. Date: Aug. 8, 2024

(54) Biological Patch and Fixation Device for Tendon Repair

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 18/434,530

(22) Filed: Feb. 6, 2024

Related U.S. Application Data
(60) Provisional application No. 63/483,703, filed on Feb. 7, 2023.

(51) Int. Cl. A61F 2/08; A61F 2/00; A61L 27/18; A61L 27/34; A61L 27/54

(52) U.S. Cl. CPC A61F 2/0811; A61L 27/18; A61L 27/34; A61L 27/54; A61F 2002/0081; A61F 2002/0852; A61F 2002/0888; A61F 2220/0016; A61F 2220/0075; A61F 2240/001; A61L 2430/10

(57) ABSTRACT

A biological patch or construct used in tissue engineering for tendon repair. The patch system includes a scaffold connected to at least one fastener via a suture. The patch is delivered to a torn tendon via a delivery cannula and left over a torn tendon to assist in repair of the tendon.

This application claims priority to U.S. Provisional Application 63/483,703 filed Feb. 7, 2023.

### FIELD OF THE INVENTIONS

The inventions described below relate to the field of biological patches or constructs used in tissue engineering for tendon repair.

### BACKGROUND OF THE INVENTIONS

Regenerative tissue engineering is a dynamic field that needs solutions to achieve functional tissue regeneration. Regenerative tissue engineering often includes the use of biological patches or constructs for attachment to the torn tissue to serve as the support structure during tissue regeneration. The biological patches often consist of scaffolds that mimic the natural extracellular matrix of tendons. Effective placement and securing of the patch is necessary to promote efficient healing of the tissue.

### SUMMARY

The devices and methods described below provide for improved healing of a torn tendon using a biological patch or construct to facilitate the regeneration of damaged tendons. The patch or construct includes a scaffold that serves as the extracellular matrix of the native tissue for support during the tissue regeneration. The patch or construct is attached via at least one fastener to the desired treatment location within the patient. The fastener includes channels or openings (vents) to facilitate with the integration onto the surrounding bone tissue and promote vascularization of the patch to the tendon. The fasteners are connected to the patch or construct with sutures to enhance the stability of the tissue to increase the tissue integration for healing.

### BRIEF DESCRIPTION OF THE DRAWINGS

FIG. 1 illustrates the shoulder of a patient with an area requiring surgical intervention.

FIG. 2 is a view of a regenerative patch or construct system for use in tendon repair.

FIGS. 3 and 4 are exploded view of the regenerative patch or construct system of FIG. 2.

FIG. 5 is an exploded view of another patch or construct system.

FIG. 6 is a top view of the patch system of FIG. 5 with a cross sectional view of the patch along section A-A.

FIG. 7 is a cross-sectional view of the scaffold and fastener when placed within the body of a patient.

FIG. 8 is a construct with an alternative fastener.

FIGS. 9 and 10 are an alternative scaffold.

FIG. 11 is a fastener that is a tack.

FIG. 12 is an alternative fastener that is a staple.

### DETAILED DESCRIPTION OF THE INVENTIONS

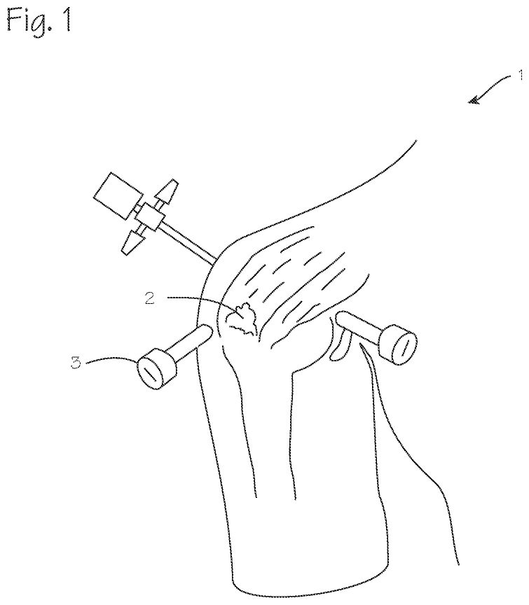

FIG. 1 illustrates a patient 1 with a tendon tear 2 in the shoulder that necessitates surgical intervention with a regenerative patch or construct system. A delivery cannula 3 has been inserted into the shoulder, with the distal end of the delivery cannula proximate the torn tendon. The delivery cannula is for introduction of a regenerative patch construct system to the tendon repair site.

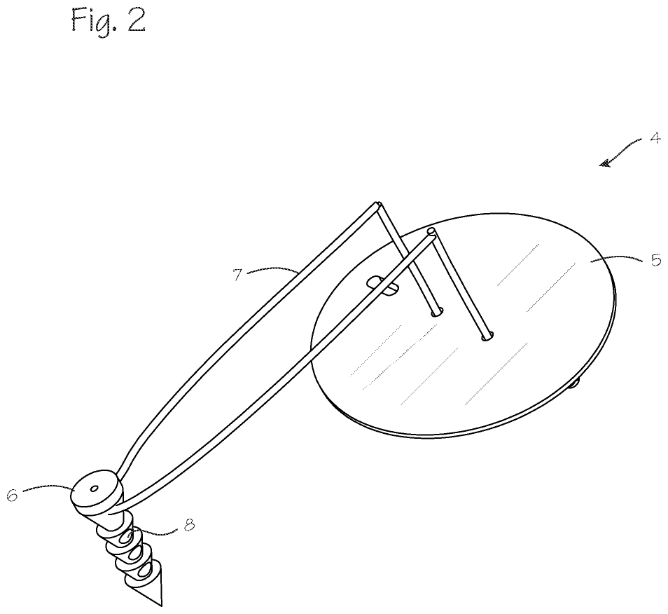

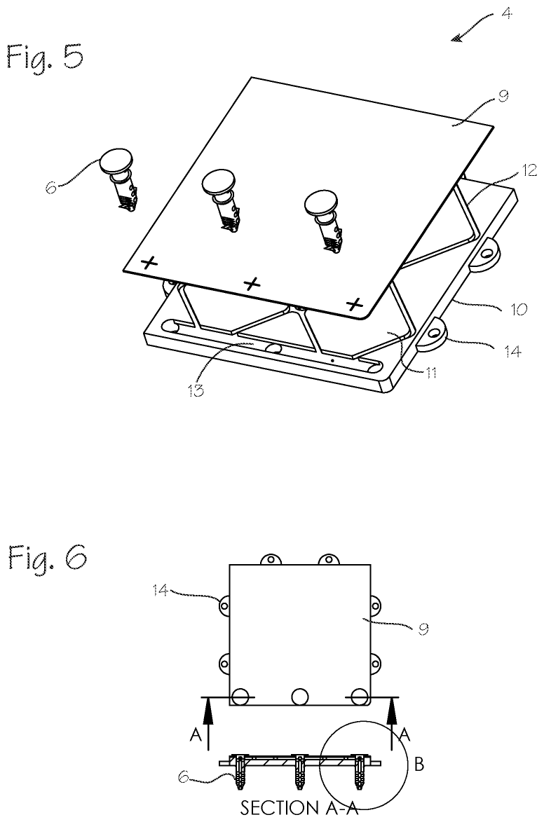

FIG. 2 is a view of a regenerative patch or construct system 4 for use in tendon repair. The system includes a scaffold 5 connected to at least one fastener 6 via a suture 7. FIG. 2 illustrates a circular scaffold, however, it can be of any shape. The scaffold serves as the extracellular matrix of the native tissue for support during the tissue regeneration. The scaffold is rollable so that it can be advanced through a delivery cannula to be delivered to a desired repair site within a patient. The fastener includes channels or ports 8 that help the fastener integrate onto bone tissue and also allow fluid to vent and flow through the channels or ports. The fastener is connected to the scaffold via sutures. Alternatively, the fluid path of the bone marrow may be from a fastener advanced directly through the scaffold as illustrated in FIG. 5. Once inserted to the desired treatment site, fluid such as bone marrow blood may be flowed from the fastener to the scaffold over a fluid path via the suture. The blood bone marrow assists in healing of the tendon to shorten the recovery period for a patient. The system may include more than one fastener.

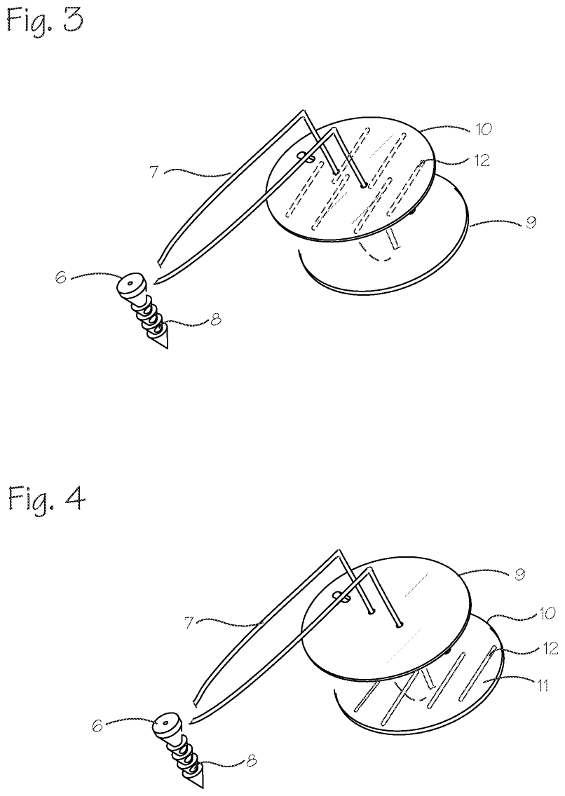

FIGS. 3 and 4 are exploded views of the scaffold of the regenerative patch or construct system of FIG. 2. The scaffold includes a first sheet 9 and an opposing second sheet 10. The second sheet has a first surface 11 and a second surface. The first surface includes microchannels 12 for introduction and flowing of fluids through the scaffold and to the tendon. The first sheet is positioned in opposition to the second sheet to enclose the microchannels between the first sheet and the second sheet within the scaffold. FIGS. 3 and 4 illustrate the first and second sheets each alternatively positioned on the top and bottom of the scaffold. The scaffold may be a unitary design where the first sheet and the second sheet are of a unitary construction. Alternatively, the scaffold may combine distinct sheets into a construction of two different sheets.

FIG. 5 is an exploded view of another patch or construct system. The system includes a square shaped scaffold with a plurality of fasteners 6. The scaffold includes a first sheet 9 and a second sheet 10. The second sheet includes microchannels 12 and a reservoir 13 for introduction and flowing of fluids through the scaffold. The first sheet is positioned in opposition to the second sheet to enclose the microchannels and reservoir between the first sheet and the second sheet within the scaffold. The second sheet may also optionally contain grommets 14 along the perimeter of the second sheet to further assist in securing or placing the scaffold over a tendon. At least one fastener pierces through the first sheet and the second sheet to secure the construct to a desired location over a tendon that requires repair. The fastener may be a tack or screw that secures the scaffold to the tendon. The fastener may also be connected to the scaffold via a thread or suture. The fastener is a channeled tack that contains holes along the stem of the tack to allow for fluid flow between the tack and the scaffold. Optionally, as seen in FIG. 2, the fastener does not have to be secured through the scaffold and can be secured at a location not on the scaffold.

FIG. 6 is a top view of the patch system of FIG. 5 with a cross sectional view of the patch along section A-A. This scaffold is square shaped. The second sheet has grommets that project from the perimeter of the second sheet to allow for additional connections of the construct to the tendon. The cross-sectional view illustrates the patch assembly with fasteners projecting through the scaffold.

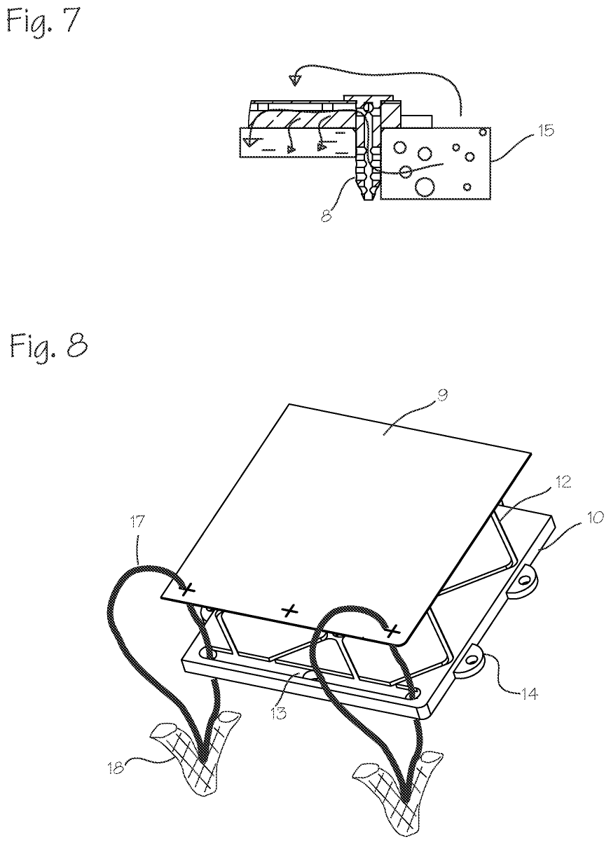

FIG. 7 is a cross-sectional view of the scaffold and fastener when placed within the body of a patient and the flow path of fluid when the fastener is positioned. The construct and fastener are positioned over a torn tendon 2. The fastener contains channels or ports 8 on the stem of the fastener. Fluid, such as bone blood marrow, 15 is introduced through the channels and allows access of blood bone marrow to inflow though the fastener to flow through the microchannels within the scaffold. The blood bone marrow assists in healing of the tendon to shorten the recovery period for a patient.

FIG. 8 is a construct with an alternative fastener. The fastener is a monofilament suture 16 and anchor 17 The monofilament suture is secured to the scaffold and also the anchor. The anchor and sutures can both be comprised of any woven wicking material. Blood bone marrow is vented to the patch via the suture to increase the healing effect of the construct on the tendon.

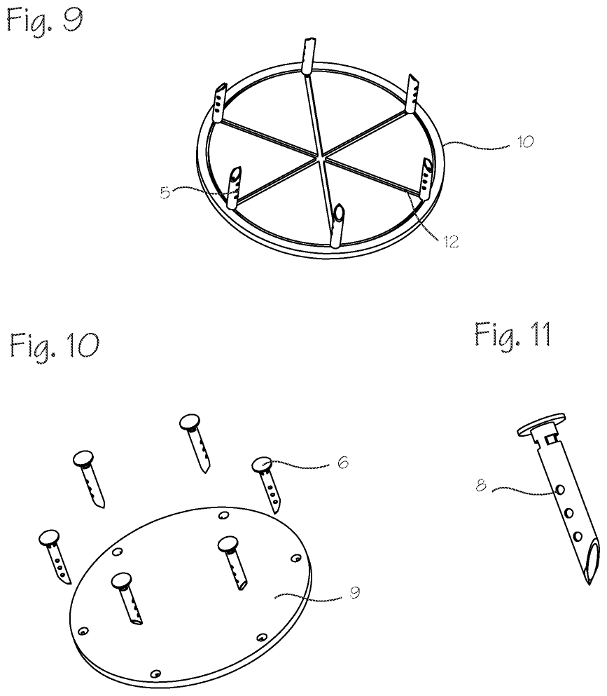

FIGS. 9 and 10 are an alternative scaffold. The first 9 and second sheets 10 are circular. The microchannels 12 on first face of the second sheet radiate from a central point of the second surface and extend to the circumference of the circular sheet. The first sheet and second sheet are mated together and secured via a tack to each other and then to the tendon of a patient.

FIG. 11 is a fastener that is a tack. The tack has a stem with channels or ports 5 that allow the flow of blood bone marrow through the tack to the reservoir and microchannels of the second sheet.

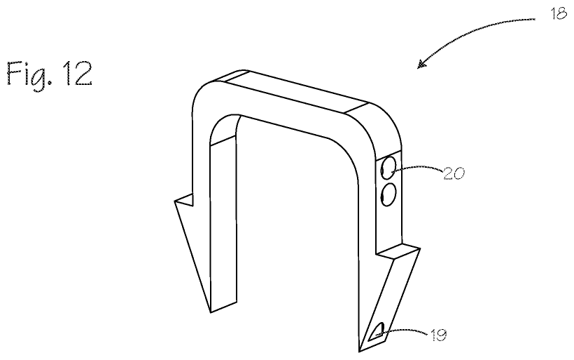

FIG. 12 is an alternative fastener 18 that is a staple. The fastener includes a fluid inlet port and 19 an outlet port 20 on the side prongs of the staple. When the fastener is positioned over a scaffold within a patient, fluid such as bone blood marrow can be introduced through the inlet port and flowed through the outlet port to flow through the mirochannels on the top surface of the second sheet.

The suture can be a monofilament or multifilament (braided) non absorbable wicking suture with sufficient tensile strength to affect a rotator cuff or tendon repair through the scaffold. The suture may be coated with antimicrobial properties to prevent infections. The suture provides mechanical stability and support and positioning of the patch during healing process.

The scaffold acts as a frame for cell attachment to enhance the healing process during tissue regeneration. The scaffold is made from natural or synthetic materials, proteins, glycoproteins, proteoglycans or collagens. It has an impermeable structure to increase the tissue integration for healing. The scaffold mimics the natural extracellular matrix of the tendon. The scaffold can include growth stimulating factors to increase cellular response. The scaffold can be formed by electrospinning, 3D printing or manually bonding or braiding by aligning and compressing sheets together to create a scaffold. For example, the scaffold may be formed by PLLA electrospinning and include a poly(l-lactide-co-ε-caprolactone) scaffold frame. The scaffold may also be compression molded poly(l-lactide-co-ε-caprolactone), woven PLLA, PGA, PLGA or other fibers, or any foamed biopolymer or collagen with a polymer component. The scaffold may also be formed by any template assisted method, drawing method, self-assembly technique, phase separation technique or other coaxial electrospinning.

The second sheet is an impermeable sheet comprising a less porous substrate. It can be made of resilient non porous and less porous materials such as Polydioxanone, Poly-L-lactide, Poly-D-lactide, Poly-DL-lactide, Polyglycolide, ε-Caprolactone, poly(l-lactide-co-ε-caprolactone), Polyethylene glycol, cellulose or gelatin.

The first sheet maintains fluid or bone marrow blood between the first sheet and second sheet within the patch and in contact with the desired body part or tendon. The first sheet is made of a permeable material or porous substrate such as PLLA electrospun, with a poly(l-lactide-co-ε-caprolactone) frame, or compression molded poly(l-lactide-co-ε-caprolactone) Woven PLLA, PGA, PLGA or other fibers, or any appropriate foamed biopolymer or collagen with a polymer component.

The first sheet collagen coating is made from an aligned nanocollagen, bovine collagen, porcine derived collagen, or non-animal recombinant collagen.

The first and second sheets may also include bioactive material coatings and additives such as BMP (bone morphogenic proteins) bone fibers, hyaluronic acid, PRP (platelet rich plasma) either in dehydrated form or in a coating or hydrogel.

The fluid path from the hard or soft anchor can be from the bone anchor, through the patch construct, or through a multifilament non-absorbable braided wicking suture with sufficient tensile strength to affect a rotator cuff or tendon repair, and through a fluid shunt tube in addition to the structural suture made of a bioabsorbable material or implantable non-bioabsorbable material.

In use, the desired surgical site is prepared and incisions are made to create portal access to a torn tendon in a patient. A delivery cannula is inserted into a first incision site to allow introduction of the patch through an obturator tip to the repair site. An introducer is used to deliver a patch through the delivery cannula. The patch is positioned along the tendon surface and held in place by the introducer. An inserter is introduced through a second incision site to an anchor fixation position on the edge of the patch and then the patch is secured to the tendon. At least one fastener is secured to the tendon to secure the patch. Once the patch is secure, the introducer is removed from the patient. When the channeled fastener is positioned within the tendon the channeled fasteners then flow fluid such as bone marrow blood to the patch via the suture. The flow action is accomplished by a wicking process which relies on a capillary action as the capillary helps draw the fluid into the sheet over the suture. The flow action advances from the fastener, down or over the suture to the scaffold, and then from the microchannels within the scaffold to the tendon. Additionally, the patch can be infused with additional biologic fluid if needed once the fastener is secured.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A regenerative patch system for treating a tendon comprising:
a rollable scaffold comprising a first sheet and a second sheet opposing the first sheet, the second sheet having a first surface and a second surface wherein the first surface includes microchannels to flow fluid through the second surface and wherein the first sheet and the second sheet enclose the microchannels between the two sheets;
a fastener having fluid channels; and
a suture securing the fastener to the rollable scaffold, the patch system adapted to flow fluid from the fastener via the suture, to the patch.

2. The regenerative patch system of claim 1 the first sheet is coated with collagen.

3. The regenerative patch system of claim 1 wherein the second sheet is comprised of a material selected from the consisting of Polydioxanone, Poly-L-lactide, Poly-D-lactide, Poly-DL-lactide, Polyglycolide, ε-Caprolactone, poly(l-lactide-co-ε-caprolactone), Polyethylene glycol, cellulose and gelatin.

4. The regenerative patch system of claim 1 wherein the suture is a monofilament.

5. The regenerative patch system of claim 1 wherein the scaffold is formed by electrospinning.

6. The regenerative patch system of claim 5 wherein the scaffold comprises poly(l-lactide-co-ε-caprolactone).

7. The regenerative patch system of claim 1 wherein the fastener is a tack.

8. The regenerative patch system of claim 1 wherein the fastener is a staple.

9. The regenerative patch system of claim 1 wherein the first and second sheets further comprise a bioactive material coating selected from the group comprising bone morphogenic proteins, bone fibers, hyaluronic acid, platelet rich plasma and a hydrogel.

10. The regenerative patch system of claim 1 wherein the second surface of the second sheet further includes a reservoir for collection of fluid.

11. A method of inserting a regenerative patch system over a patient's tendon comprising the steps:
providing a regenerative patch for placement over a torn tendon, said regenerative patch comprising:
a rollable scaffold comprising a first sheet and a second sheet opposing the first sheet, the second sheet having a first surface and a second surface wherein the first surface includes microchannels to flow fluid through the second surface and wherein the first sheet and the second sheet enclose the microchannels between the two sheets;
a fastener having fluid channels; and
a suture securing the fastener to the rollable scaffold, the suture adapted to flow fluid from the fastener to the patch;
inserting a delivery cannula having a proximal end and a distal end into a first incision site;
inserting an introducer with a proximal end and a distal end through the delivery cannula;
inserting the distal end of the cannula and the distal end of the introducer to the tendon repair site adjacent a torn tendon and positioning the patch along the tendon surface and holding the patch in place by the introducer;
creating a second incision and introducing an inserter to an anchor fixation position on the edge of the patch and then securing the patch to the tendon via at least one fastener;
removing the introducer;
flowing bone marrow blood to the patch via the suture.
